(19) United States
(12) Patent Application Publication
(10) Pub. No.: US 2020/0270555 A1
(43) Pub. Date: Aug. 27, 2020

(54) Gradient Microfluidic Devices And Uses Thereof

(71) Applicant: President and Fellows of Harvard College, Cambridge, MA (US)

(72) Inventors: Donald E. Ingber, Boston, MA (US); Kyung-Jin Jang, Brookline, MA (US); Daniel Levner, Brookline, MA (US); Norman Wen, Newton, MA (US)

(21) Appl. No.: 15/781,049

(22) PCT Filed: Nov. 30, 2016
(86) PCT No.: PCT/US16/64179

§ 371 Date: Jun. 1, 2018

Related U.S. Application Data
(60) Provisional application No. 62/263,386, filed on Dec. 4, 2015.

(51) Int. Cl. C12M 3/00; C12M 3/06; C12M 1/00; C12M 1/34; G01N 33/50

(52) U.S. Cl. CPC C12M 21/08; C12M 23/16; G01N 33/5044; C12M 41/30; C12M 23/34

(57) ABSTRACT

A device simulates a function of a tissue and includes a first structure defining a first chamber, a second structure defining a plurality of second chambers, and a membrane located at an interface region between the first chamber and the plurality of second chambers. The second structure extends along the first chamber. Each of the second chambers has a fluid therein, with each fluid having an agent of a different concentration and/or flowing at a different flow rate. The membrane, which separates the first chamber from the plurality of second chambers, has cells adhered on a first side facing toward the first chamber and on a second side facing toward the plurality of second chambers.

### CROSS-REFERENCE TO RELATED APPLICATION

This application claims priority to and benefit of U.S. Provisional Patent Application Ser. No. 62/263,386, filed on Dec. 4, 2015, which is hereby incorporated by reference herein in its entirety.

### FIELD OF THE INVENTION

The present invention relates generally to devices and methods for creating varying cellular microenvironments, and, more particularly, to simulating a tissue function on a chip.

### BACKGROUND OF THE INVENTION

The kidney is an incredibly intricate organ, and the nephron, its functional unit, is composed of over 10,000 cells with many different cell types and variants. The main functions of the kidney are filtration, reabsorption, and secretion to maintain the human body's homeostasis. The distribution of nephron's cell types and variants are highly related to the location of the cells along the nephron. At the broadest scale, the nephron is separated into four main sections: the proximal convoluted tubule, the loop of Henle, the distal convoluted tubule, and the collecting tubule, with each segment having unique architecture, function, and osmotic pressure. Therefore, it is very complicated to mimic the kidney's tubule environment in an in vitro model.

To better recapitulate the nephron in vitro, it is desirable to recreate the varying cellular microenvironment that the kidney cells experience. This microenvironment should help drive or maintain cellular differentiation, thereby improving cellular function.

### SUMMARY OF THE INVENTION

According to one aspect of the present invention, a device for simulating a function of a tissue includes a first structure defining a first chamber, and a second structure defining a plurality of second chambers extending along the first chamber, wherein each of the second chambers has a fluid therein. Each fluid has an agent of a different concentration and/or flowing at a different flow rate. The device further includes a membrane located at an interface region between the first chamber and the plurality of the second chambers. The membrane has cells adhered on a first side facing toward the first chamber and on a second side facing toward the plurality of second chambers. The membrane separates the first chamber from the plurality of the second chambers.

According to another aspect of the present invention, a device for simulating a function of a tissue includes a first structure defining a first chamber along an axis, and a second structure defining a plurality of second chambers along the axis, each second chamber intersecting the first chamber and having a fluid therein. The fluid in each second chamber has an agent of a different concentration and/or flowing at a different flow rate. The device further includes a membrane located at an interface region between the first chamber and the plurality of the second chambers, the membrane having cells adhered on a first side facing toward the first chamber and on a second side facing toward the plurality of second chambers. The membrane separates the first chamber from the plurality of the second chambers.

According to yet another aspect of the present invention, a device for simulating a function of a tissue include a first structure defining a first chamber, and a second structure defining a second chamber, the second chamber being coupled to a gradient generator. The device further includes a membrane located at an interface region between the first chamber and the second chamber, the membrane having cells adhered on a first side facing toward the first chamber and on a second side facing toward the second chamber. The membrane separates the first chamber from the second chamber.

According to yet another aspect of the present invention, a method for simulating a function of a tissue includes (a) providing a device. The device includes (i) a first structure defining a first chamber, and (ii) a second structure defining a plurality of second chambers extending along the first chamber, wherein each of the second chambers has a fluid therein, each fluid having an agent of a different concentration. The device further includes (iii) a membrane located at an interface region between the first chamber and the plurality of the second chambers, the membrane having kidney epithelial cells adhered on a first side facing toward the first chamber and on a second side facing toward the plurality of second chambers. The membrane separates the first chamber from the plurality of the second chambers. The method further includes (b) flowing the fluid in the first chamber and the second chambers.

According to yet another aspect of the present invention, a method for simulating a function of a tissue includes (a) providing a device. The device includes (i) a first structure defining a first chamber along an axis, and (ii) a second structure defining a plurality of second chambers along the axis, each second chamber intersecting the first chamber and having a fluid therein. The fluid in each second chamber has an agent of a different concentration. The device further includes (iii) a membrane located at an interface region between the first chamber and the plurality of the second chambers, the membrane having kidney epithelial cells adhered on a first side facing toward the first chamber and on a second side facing toward the plurality of second chambers. The membrane separates the first chamber from the plurality of the second chambers. The method further includes (b) flowing the fluid in the first chamber and the second chambers.

According to yet another aspect of the present invention, a device is directed to testing agents at different concentrations, and includes a first structure defining a first chamber. The device further includes a plurality of second chambers extending outward along the first chamber, each of the second chambers including a fluid therein and being in fluidic communication with the first chamber, each fluid including an agent of a different concentration. The device also includes a membrane located at an interface region between the first chamber and the plurality of the second chambers, the membrane including cells adhered on a first side facing toward the first chamber and a second side facing toward the plurality of second chambers, the membrane separating the first chamber from the plurality of the second chambers.

According to yet another aspect of the present invention, a device is directed to testing agents at different concentrations, and includes a first structure defining a first chamber along an axis. The device further includes a plurality of second chambers along the axis, each second chamber intersecting the first chamber and including a fluid therein, the fluid in each second chamber including an agent of a different concentration. The device also includes a membrane located at an interface region between the first chamber and the plurality of the second chambers, the membrane including cells adhered on a first side facing toward the first chamber and a second side facing toward the plurality of second chambers, the membrane separating the first chamber from the plurality of the second chambers.

According to yet another aspect of the present invention, a device is directed to exposing cells to gradients, and includes a first structure defining a first chamber. The device further includes a second structure defining a second chamber, the second chamber being coupled to a gradient generator. The device also includes a membrane located at an interface region between the first chamber and the second chamber, the membrane including cells adhered on a first side facing toward the first chamber and a second side facing toward the second chamber, the membrane separating the first chamber from the second chamber.

According to yet another aspect of the present invention, a method is directed to testing agents at different concentrations. The method includes (a) providing a device with (i) a first structure defining a first chamber, (ii) a plurality of second chambers extending outward along the first chamber, each second chamber of the plurality of second chambers including a fluid therein and being in fluidic communication with the first chamber, each fluid including an agent of a different concentration, and (iii) a membrane located at an interface region between the first chamber and the plurality of the second chambers, the membrane including cells adhered on a first side facing toward the first chamber and a second side facing toward the plurality of second chambers, the membrane separating the first chamber from the plurality of the second chambers. The method further includes (b) flowing the fluid in the first chamber and the second chambers.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a schematic diagram showing one embodiment of a lateral gradient chip with three vascular channels underlying a single epithelial channel.

FIG. 2 is a schematic diagram showing one embodiment of a lateral gradient chip with a gradient generator.

FIG. 3 is a schematic diagram showing one embodiment of a longitudinal gradient chip with three channels or “zones” along the length.

FIG. 4 is a schematic diagram showing one embodiment of a longitudinal gradient chip comprising a gradient generator.

FIG. 5 is a schematic diagram showing a gradient chip, according to an alternative embodiment.

FIG. 6A is a schematic illustration showing a mixer network with a one-dimensional (“1D”) concentration gradient.

FIG. 6B is a schematic illustration showing a mixer network with a two-dimensional (“2D”) concentration gradient.

FIG. 7A is a schematic illustration showing a T-junction.

FIG. 7B is a schematic illustration showing a Y-junction.

FIG. 7C is a schematic illustration showing a Flow splitter.

FIG. 8A is a schematic illustration showing a pressure balance with a 1D concentration gradient.

FIG. 8B is a schematic illustration showing a pressure balance with a 2D concentration gradient.

FIG. 9A is a schematic illustration showing a hydrogel/extracellular matrix (“ECM”) with a 1D concentration gradient.

FIG. 9B is a schematic illustration showing a hydrogel/ECM with a 2D concentration gradient.

FIG. 9C is a schematic illustration showing a hydrogel/ECM with a tree-dimensional (“3D”) concentration gradient.

FIG. 10 is a schematic illustration showing an open-cell configuration with a 2D concentration gradient.

FIG. 11 is an isometric view of an organ-on-chip (“OOC”) device, according to an alternative embodiment.

FIG. 12 is a cross-sectional perspective front view representation along sectional lines 12-12 of FIG. 11.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

While this invention is susceptible of embodiment in many different forms, there is shown in the drawings and will herein be described in detail preferred embodiments of the invention with the understanding that the present disclosure is to be considered as an exemplification of the principles of the invention and is not intended to limit the broad aspect of the invention to the embodiments illustrated. For purposes of the present detailed description, the singular includes the plural and vice versa (unless specifically disclaimed); the words “and” and “or” shall be both conjunctive and disjunctive; the word “all” means “any and all”; the word “any” means “any and all”; and the word “including” means “including without limitation.”

### Definitions

The term “microfluidic” as used herein relates to components where a moving fluid is constrained in or directed through one or more channels in which one or more dimensions are 1 millimeter (“mm”) or smaller (microscale). Microfluidic channels may be larger than microscale in one or more directions, though the channel(s) will be on the microscale in at least one direction. In some instances, the geometry of a microfluidic channel is configured to control the fluid flow rate through the channel (e.g. increase channel height to reduce shear). Microfluidic channels are formed of various geometries to facilitate a wide range of flow rates through the channels.

“Channels” are pathways (whether straight, curved, single, multiple, in a network, etc.) through a medium (e.g., silicon) that allow for movement of liquids and gasses. Channels, thus, connect other components, i.e., keep components “in communication” and more particularly, “in fluidic communication,” and still more particularly, “in liquid communication.” Such components include, but are not limited to, liquid-intake ports and gas vents. Microchannels are channels with dimensions less than 1 mm and greater than 1 micron.

As used herein, the phrases “connected to,” “coupled to,” “in contact with,” and “in communication with” refer to any form of interaction between two or more entities, including mechanical, electrical, magnetic, electromagnetic, fluidic, and thermal interaction. For example, in one embodiment, channels in a microfluidic device are in fluidic communication with cells and (optionally) a fluid source, such as a fluid reservoir. Two components are coupled to each other even if they are not in direct contact with each other. For example, two components are coupled to each other through an intermediate component (e.g., tubing or other conduit).

In some aspects, methods for creating varying cellular microenvironments for in vitro or organ-on-chip models are described herein. These methods and/or models can be used particularly for a kidney-on-chip, to gain improved cellular differentiation and function, but they can also be used for other organs-on-chips (e.g., not limited to airway, liver, etc.). For example, microfabrication techniques can be adapted to enable precise control of tissue organization and cell positioning in highly structured scaffold. Microfluidics tools enable fine control of dynamic fluid flows and pressure on the micrometer scale; therefore, it is possible to create a microenvironment that presents cells with organ relevant chemical gradients and mechanical cues that promote cells to express a more differentiated ordinary phenotype. This approach can contribute to restructure renal tubular organization and functional complexity in a chip, which has an in-vivo-like microarchitecture and microenvironmental signals.

### I. Lateral Gradient Chips

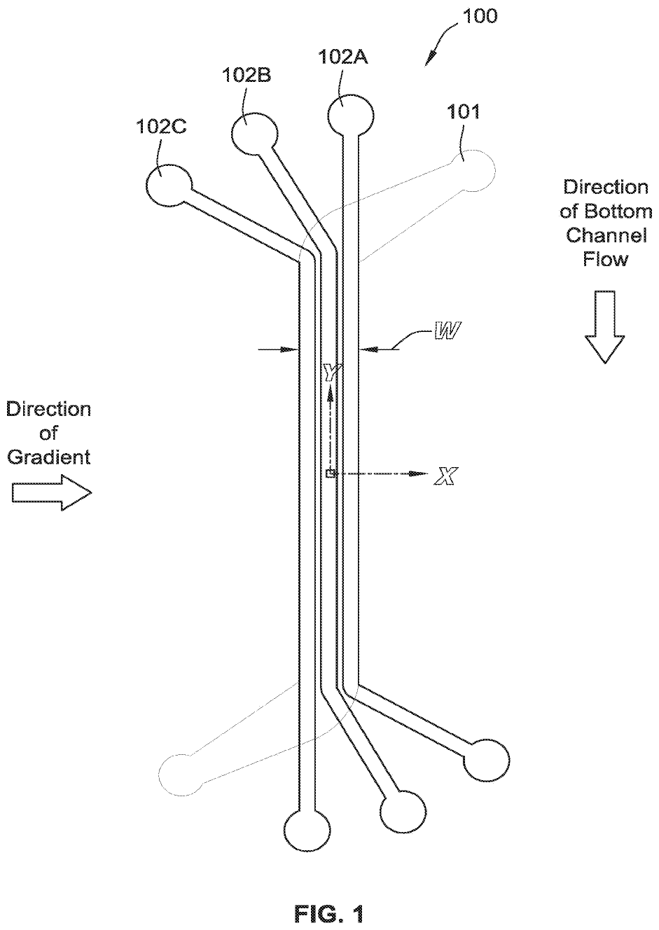

FIG. 1 illustrates an embodiment of an organ-on-chip 100 designed such that each epithelial channel 101 corresponds to a number of side-by-side vascular channels 102A, 102B, 102C (collectively referred to as “vascular channels 102”). By perfusing each of the vascular channels 102 differently (e.g., with different media, at different pressures of flow rates, etc.), the effect of the microenvironment effect can be studied. As a particular example, in reference to a human kidney, the three vascular channels 102 are perfused with media adjusted to three different salinity or osmolarity levels to explore the effect of this variation on kidney epithelial cells. The effect on both the epithelial and endothelial cells are subsequently evaluated by examining, for example, cell morphology and/or by immunohistochemically staining the cells.

In some embodiments, each epithelial channel 101 corresponds to two or more of the side-by-side vascular channels 102. Each of the vascular channels 102 is perfused with different media, at different pressures, and/or different flow rates. Thus, cells in the epithelial channel 101 are subjected to a gradient across a width W of the channel.

A “lateral gradient” configuration is useful, for example, as a research tool to evaluate the specific effect of the microenvironment on the various cells. In particular, this approach is used to identify or optimize conditions that would be used in studies that do not involve gradient chips, or in studies that use longitudinal gradient chips, which will be described below.

As a variation of the lateral gradient chip, the set of lateral channels 102 is replaced with a gradient generator that is adapted to generate a gradient across the opposing channel. According to some embodiments, the gradient generator is one known in the microfluidic art and described, for example, by Alicia G. G. Toh. et al. in Microfluidics and Nanofluidics (DOI 10.1007/s10404-013-1236-3, “Engineering Microfluidic Concentration Gradient Generators for Biological Applications,” ISSN 1613-4982, published online on Jul. 24, 2013), the content of which is incorporated herein by reference in its entirety. The most suitable design is selected for a given implementation.

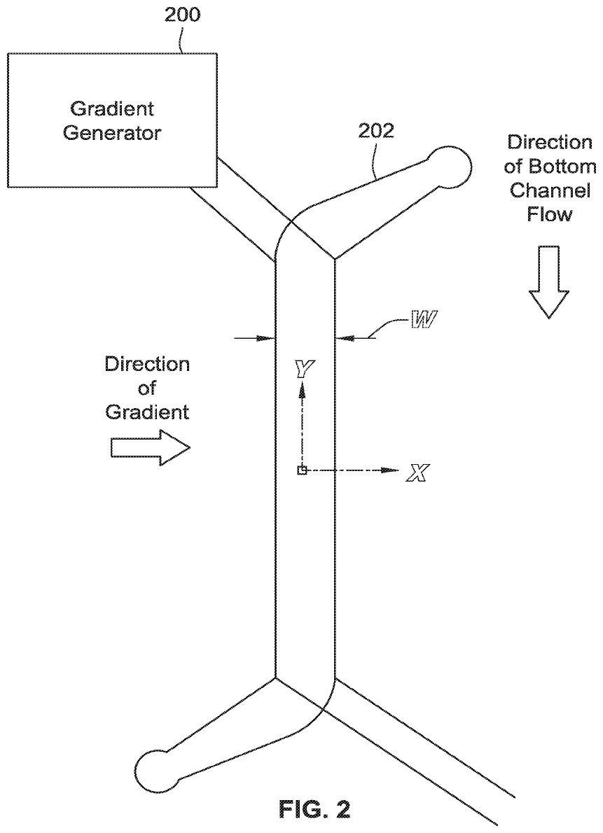

FIG. 2 illustrates an embodiment involving an exemplary gradient generator 200. An art-recognized gradient generator 200 is coupled to one end of a vascular channel 202, creating a gradient across the width W of the channel 202. Thus, cells in the “epithelial” channel are subjected to the gradient.

In addition to exploring effects of salinity or osmolarity in the kidney, the gradient chip is used, for example, for exploring oxygen gradients in the liver, variations along the airway, or the segmentation of the small or large intestine.

An additional or alternative use of the gradient chip is related to a study of tubule-tubule interaction. In such embodiments, multiple lateral channels represent different nephrons or different parts of the same nephron. In turn, a common opposing channel (representing vascular or interstitial fluid) accounts for the tubule-to-tubule coupling through the respective liquid. These embodiment are useful in modeling the loop of Henle, wherein the ascending and descending portions interact with each other.

### II. Longitudinal Gradient Chips

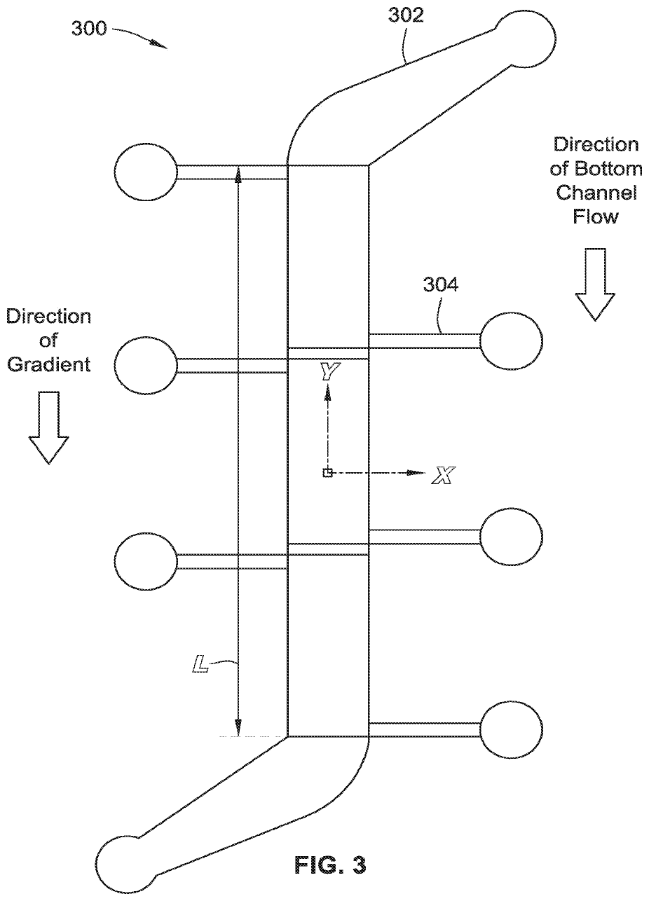

FIG. 3 illustrates an embodiment of a longitudinal gradient chip 300 in which a single epithelial channel 302 is opposed (or intersected) by multiple vascular channels 304 along its length. Similarly to the lateral design, the multiplicity of channels 304 is used to create a variety of cellular microenvironments. In this case, however, the change is along the direction of flow. This is intended as a direct analog to the variation of environment along the length of the nephron, the liver sinusoid, the airway, or the intestines. Accordingly, the media, flow conditions, and/or mechanical actuation is varied in each of the created zones. Each vascular channel 304 is perfused with different media, at different pressures, and/or different flow rates. Thus, cells in the epithelial channel 302 are subjected to a gradient along a length L of the channel 302.

In some embodiments, the multiplicity of channels 304 is replaced or supplemented with a smooth gradient, including gradient generator designs known in the art and/or any other suitable designs.

Optionally, in one alternative embodiment the epithelial channel 302 is a common hepatocyte channel, and the vascular channels 304 are Liver Sinusoidal Endothelial Cells (“LSEC”) vascular channels. In this embodiment, the channel structure recapitulates an oxygen gradient that occurs within the in vivo liver sinusoid, between the periportal and the perivenous regions. For example, the LSECs in all three channels 304 are used, but media is perfused with different concentrations of dissolved oxygen.

Similarly, in a further example, the channels 302 and 304 are used to model the intestine, which also has different regions with differing oxygen concentrations. To illustrate the use of the oxygen gradient with the intestine, the common channel 302 is used for the vasculature and the different side channels 304 are used to represent different regions of the intestinal track. For example, the side channels 304 are seeded with different epithelial cells, and are, optionally, used with different media or are used to dissolve an oxygen concentration.

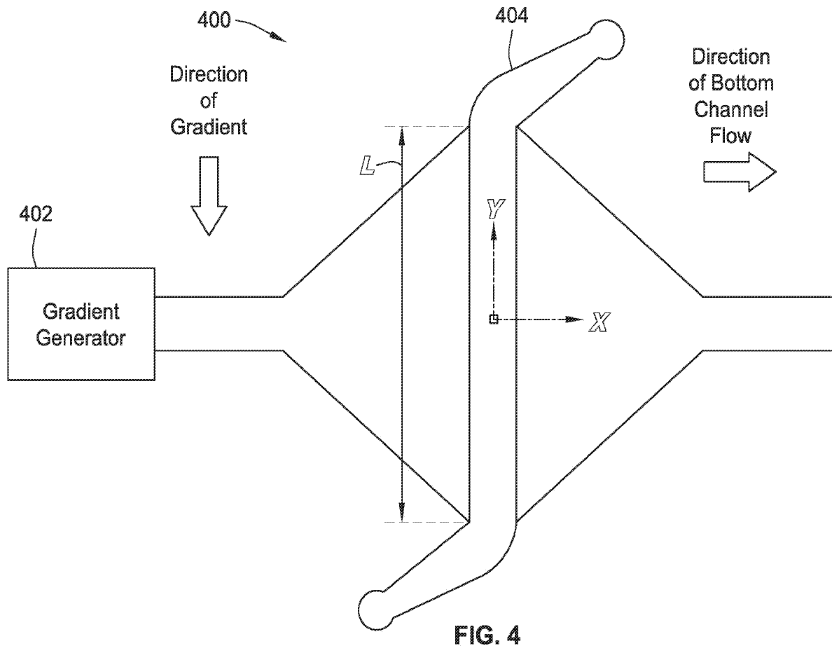

FIG. 4 illustrates an embodiment of a longitudinal gradient chip 400 that includes a gradient generator 402. For example, the gradient generator 402 is an art-recognized gradient generator, which is coupled to a vascular channel 404 such that a gradient is created along a length L of the channel 404. Thus, cells in the epithelial channel are subjected to the gradient.

There are many uses for the longitudinal gradient chip 400, in which the variation in microenvironment along the flow recapitulates an in-vivo property, thereby leading to better function in vitro. Some examples include variation of salinity or osmolarity along the length of the nephron, variation in oxygenation along the length of liver sinusoid, variation of environment and/or cell type along the length of the intestine, variation of environment, variation of flow characteristics, and/or variation of cell type along the airway.

In various aspects described herein, the variation in microenvironment is used to drive cellular differentiation. This variation is beneficial to differentiation of stem cells.

In other various aspects described herein, the devices described herein are used to create a gradient, e.g., in concentration, shear stress, or pressure within a channel. These devices are used to develop different types of organ chips (which are not limited to a kidney-on-a-chip).

### Alternative Embodiments/Optional Features

In addition to exploring effects of salinity or osmolarity in the kidney, either of the lateral or longitudinal gradient chip is used, for example, to explore or recapitulate oxygen gradients in the liver, variations along the airway, or the segmentation of the small or large intestine.

The gradients or varied parameters are also used to explore pathological or non-physiological conditions. The “gradient” does not have to be continuous or monotonic. For example, channel 1 has 0% oxygen, channel 2 has 100% oxygen, and channel 3 has 50% oxygen.

Additionally or alternatively, the designs are used to evaluate gradients in drug, hormone, and/or chemical concentration where fully independent chip replicates may not be necessary.

Although the examples described herein illustrate a gradient generated on the vascular side of an organ chip, other embodiments employ a gradient on the epithelial/interstitial side. Examples of a gradient generated on the vascular side of an organ chip are described in more detail in U.S. Pat. No. 8,647,861 (“the '861 patent”) (titled “Organ Mimic Device with Microchannels and Methods of Use and Manufacturing Thereof” and issued on Feb. 11, 2014) and PCT Application No. PCT/US2014/071611 (titled “Low Shear Microfluidic Devices and Methods of Use and Manufacturing Thereof” and filed on Dec. 19, 2014), the contents of each of which being incorporated herein by reference in their respective entirety.

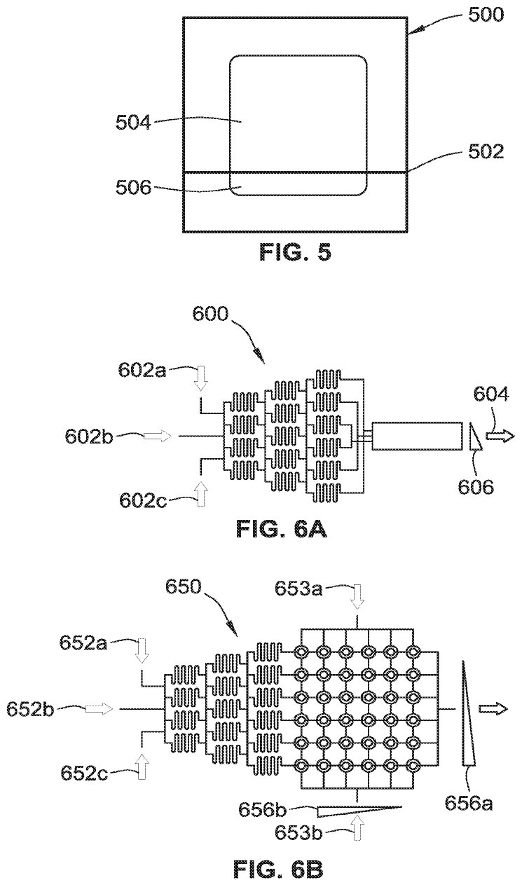

FIG. 5 illustrates another embodiment of a gradient chip 500 that includes a membrane 502, a first chamber 504, and a second chamber 506. The gradient chip 500 is configured for use with one or more channels and/or a gradient generator, as described above in reference to FIGS. 1-4.

FIGS. 6A-10 illustrate a plurality of gradient generators that can be used with any of the chips described above in reference to FIGS. 1-5. By way of example, FIGS. 6A and 6B shows “Christmas tree” mixer networks. Specifically, FIG. 6A shows a “Christmas tree” mixer network 600 with a 1D concentration gradient. Three inlet reagents 602a-602c are inputted, a single outlet flow 604 is outputted, and a concentration gradient 606 is formed. FIG. 6B shows another “Christmas tree” mixer network 650, but with a 2D concentration gradient. Specifically, the network includes three first inlet reagents 652a-652c, two second inlet reagents 653a, 653b, a single outlet flow 654, and two concentration gradient formations 656a, 656b.

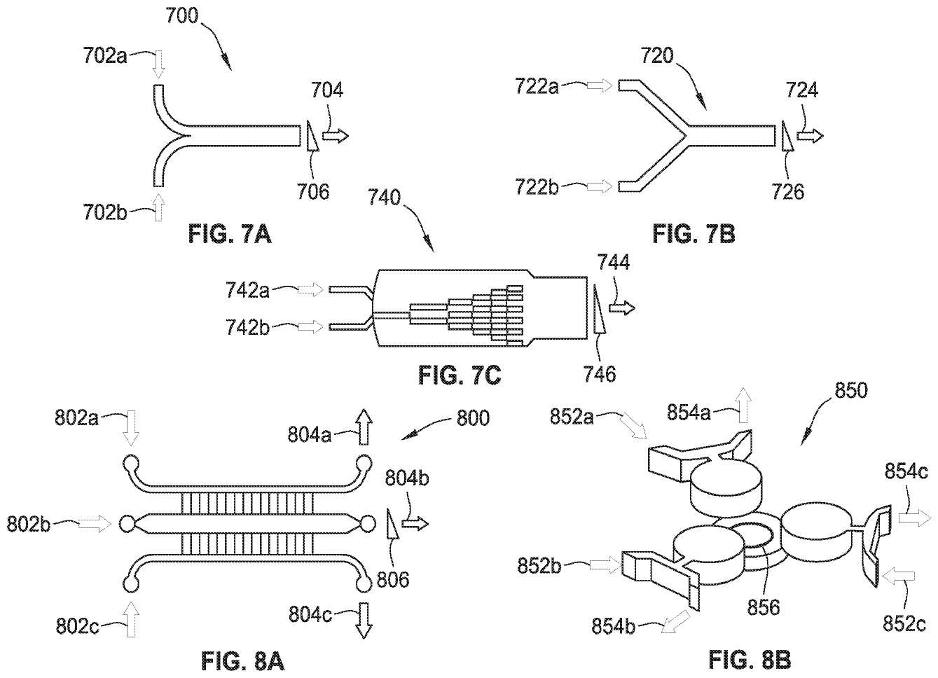

In other examples of a gradient generator, FIGS. 7A-7C shows various flow junctions and splitters. Specifically, FIG. 7A shows a T-junction 700 with two inlet reagents 702a, 702b, a single outlet flow 704, and a concentration gradient formation 706. FIG. 7B shows a Y-junction 720 with two inlet reagents 722a, 722b, a single outlet flow 724, and a concentration gradient formation 726. FIG. 7C shows a Flow splitter 740 with two inlet reagents 742a, 742b, a single outlet flow 744, and a concentration gradient formation 746.

In further examples of a gradient generator, FIGS. 8A and 8B show configurations with a different pressure balance. Specifically, FIG. 8A shows a gradient generator 800 having a pressure balance with a 1D concentration gradient. The gradient generator 800 includes three inlet reagents 802a-802c, three outlet flows 804a-804c, and a single concentration gradient 806. In another example, FIG. 8B shows a gradient generator 850 having a pressure balance with a 2D concentration gradient. The gradient generator 850 includes three inlet reagents 852a-852c, three outlet flows 854a-854c, and a concentration gradient formation 856.

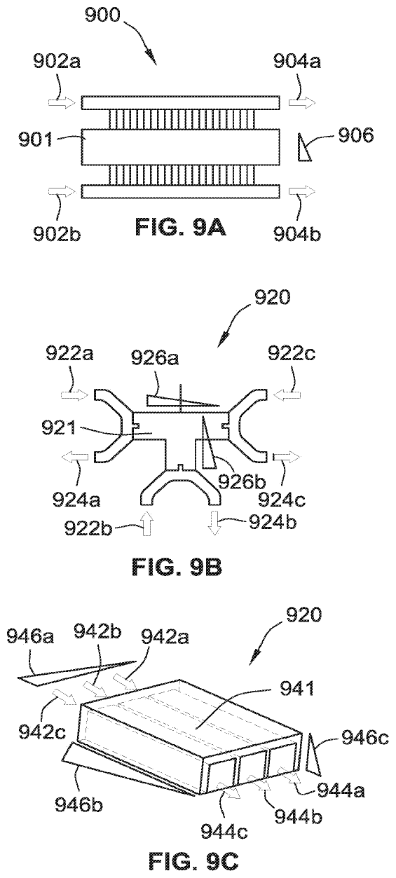

In yet further examples of a gradient generator, FIGS. 9A-9C show configuration with hydrogel and/or ECM. Specifically, FIG. 9A shows a gradient generator 900 with a hydrogel and/or ECM element 901, and further includes two inlet reagents 902a, 902b, two outlet flows 904a, 904b, and a concentration gradient formation 906. FIG. 9B shows a gradient generator 920 with a hydrogel and/or ECM element 921, three inlet reagents 922a-922c, three outlet flows 924a-924c, and two concentration gradient formations 926a, 926b. FIG. 9C shows a gradient generator 940 with a hydrogel and/or ECM element 941, three inlet reagents 942a-942c, three outlet flows 944a-944c, and three concentration gradient formations 946a-946c.

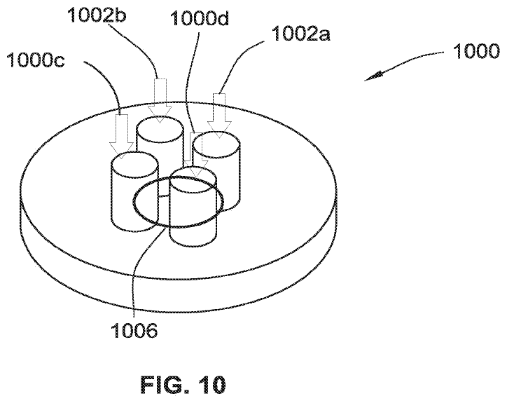

In yet another further example of a gradient generator, FIG. 10 shows an open-cell configuration (e.g., submersible probes). Specifically, a gradient generator 1000 has an open liquid well 1001, and includes four inlet reagents 1002a-1002d and a concentration gradient formation 1006.

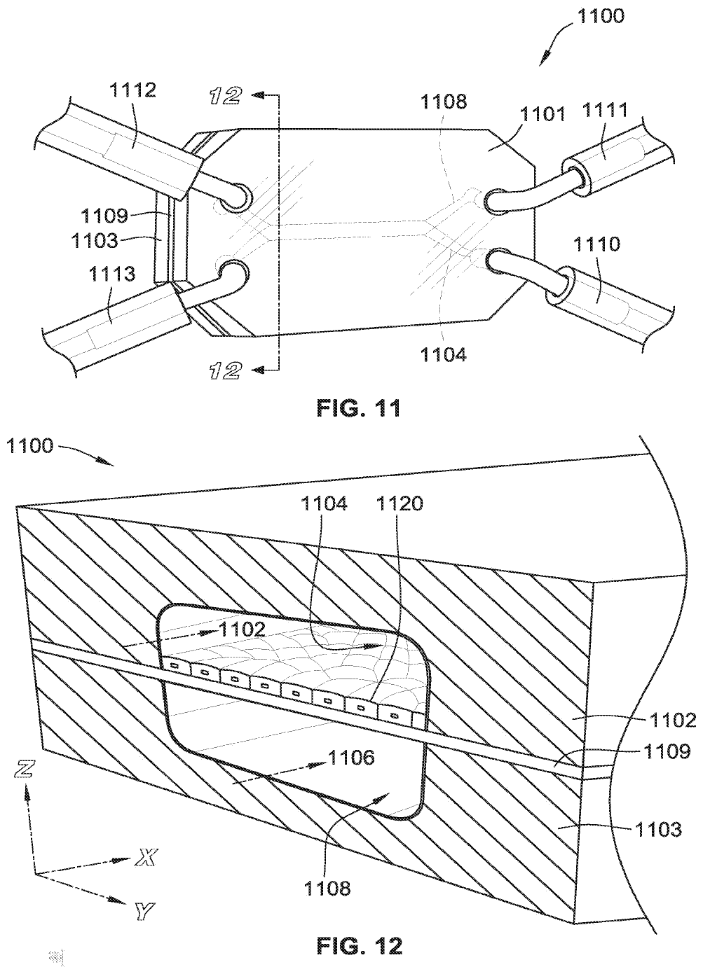

Referring to FIGS. 11 and 12, a lateral gradient chip is in the form of an OOC device 1100 that is configured typically made of a polymeric material and includes an upper body segment 1101 and a lower body segment 1103. The OOC device 1100 has a first microchannel 1104, along an X axis, and a second microchannel 1108, along the X axis and through which respective mediums flow in accordance with desired experimental use. For example, as illustrated in FIG. 12 (and assuming that the first microchannel 1104 is a top microchannel and the second microchannel 1108 is a bottom microchannel), an apical medium 1102 flows through the top microchannel 1104 and a basal medium 1106 flows through the bottom microchannel 1108. For ease of understanding, the first microchannel 1104 will be described below as being the top microchannel and the second microchannel 1108 will be described as being the bottom microchannel. However, it is understood that, according to an alternative configuration, the first microchannel 1104 is the bottom microchannel and the second microchannel 1108 is the top microchannel.

The OOC device 1100 further has a top fluid inlet 1110 and a bottom fluid inlet 1111 via which respective mediums are inserted into the respective microchannels 1104, 1108. The mediums exit from the respective microchannels 1104, 1108 via a top fluid outlet 112 and a bottom fluid outlet 1113.

The OOC device 1100 also has a barrier 1109 that separates the microchannels 1104, 1108 at an interface region. The barrier 1109 is optionally a semi-permeable barrier that permits migration of cells, particulates, media, proteins, and/or chemicals between the top microchannel 1104 and the bottom microchannel 1108. For example, the barrier 109 includes gels, layers of different tissue, arrays of micro-pillars, membranes, and combinations thereof. The barrier 1109 optionally includes openings or pores to permit the migration of the cells, particulates, media, proteins, and/or chemicals between the top microchannel 1104 and the bottom microchannel 1108. According to one specific example, the barrier 1109 is a porous membrane that includes a cell layer 1120 (shown in FIG. 4) on at least one surface of the membrane.

According to alternative embodiments, the barrier 1109 includes more than a single cell layer 1120 disposed thereon. For example, the barrier 1109 includes the cell layer 1120 disposed within the top microchannel 1104, the bottom microchannel 1108, or each of the top and bottom microchannels 1104, 1108. Additionally or alternatively, the barrier 1109 includes a first cell layer disposed within the top microchannel 1108 and a second cell layer within the bottom microchannel 1108. Additionally or alternatively, the barrier 1109 includes a first cell layer and a second cell layer disposed within the top microchannel 1104, the bottom microchannel 1108, or each of the top and bottom microchannels 1104, 1108. ECM gels are optionally used in addition to or instead of the cell layers.

Beneficially, the above-described various combinations provide for in-vitro modeling of various cells, tissues, and organs including three-dimensional structures and tissue-tissue interfaces such as brain astrocytes, kidney glomuralar epithelial cells, etc. In one embodiment of the OOC device 1100, the top and bottom microchannels 1104, 1108 generally have a length of less than approximately 2 centimeters (“cm”), a height of less than approximately 200 microns (“μm”), and a width of less than approximately 400 μm. More details in reference to other features of the OOC device 1100 are described, for example, in the '861 patent, which has been incorporated above by reference in its entirety.

The OOC device 100 is configured to simulate a biological function associated with cells, such as simulated organs, tissues, etc. One or more properties of a working medium, such as a fluid, may change as the working medium is passed through the microchannels 1104, 1108 of the OOC device 1100, producing an effluent. As such, the effluent is still a part of the working medium, but its properties and/or constituents may change when passing through the OOC device 1100.

The OOC device 1100 optionally includes an optical window that permits viewing of the medium as it moves, for example, across the cell layer 1120 and the barrier 1109. Various image-gathering techniques, such as spectroscopy and microscopy, can be used to quantify and evaluate the medium flow or analyte flow through the cell layer 1120.

According to one example, the OOC device 1100 is directed to testing agents at different concentrations. The OOC device 1100 includes a first structure in the form of the upper body segment 1101 that defines a first chamber in the form of the microchannel 1104 along the X axis. The OOC device 1100 further includes one or more second chambers extending outward along the first chamber 1104, the second chambers including the second microchannel 1108. In alternative embodiments, the OOC device 1100 includes a plurality of second microchannels 1108 and/or a plurality of first microchannels 1104. The second chambers 1108 include a fluid therein and are in fluidic communication with the first chamber 1104, each fluid including an agent of a different concentration.

In further accordance with the above example, the OOC device 110 further includes a membrane in the form of the barrier 1109 that is located at the interface region between the first chamber 1104 and the plurality of second chambers 1108. The membrane 1109 includes cells 1120 adhered on a first side facing toward the first chamber 1104. Optionally, although not illustrated, another layer of cells 1120 is also adhered on a second side of the membrane 1109 facing toward the plurality of second chambers 1108, the membrane 1109 separating the first chamber 104 from the plurality of the second chambers 1108. Optionally, the agents are drugs and the cells adhered on the first side are selected from a group consisting of kidney epithelial cells, hepatocytes, and intestinal cells.

### Exemplary Applications

In alternative embodiments, the gradient chips described herein allow a user to test chemical, osmotic, mechanical, fluidic, and/or other microenvironment gradient with different parts of an organ. The organ includes other organs in addition to or instead of a kidney tubule.

In other alternative embodiments, a lateral design allows exploration of interaction of tubules or parts of a single tubule in nephron.

In yet other alternative embodiments, the gradient chips described herein allow a user to study the mechanism of differentiation of renal tubules or other cellular systems using stem cells.

In yet other alternative embodiments, the gradient chips described herein allow the user to mimic countercurrent flow system of the kidney tubule.

In yet other alternative embodiments, the gradient chips described herein provide a high-throughput testing tool for studying drug-induced renal toxicity or renal physiology.

In yet other alternative embodiments, the gradient chips described herein are used in exploring effects of microenvironment on cellular differentiation and function, with the results potentially applied to non-gradient organ-chips.

Each of these embodiments and obvious variations thereof is contemplated as falling within the spirit and scope of the claimed invention, which is set forth in the following claims.

1. A device for simulating a function of a tissue, comprising:
a first structure defining a first chamber;
a second structure defining a plurality of second chambers extending along the first chamber, wherein each of the second chambers has a fluid therein, each fluid having an agent of a different concentration and/or flowing at a different flow rate; and
a membrane located at an interface region between the first chamber and the plurality of the second chambers, the membrane having cells adhered on a first side facing toward the first chamber and on a second side facing toward the plurality of second chambers, the membrane separating the first chamber from the plurality of the second chambers.

2. The device of claim 1, wherein the cells adhered on the first side include kidney epithelial cells.

3. A device for simulating a function of a tissue, comprising
a first structure defining a first chamber along an axis;
a second structure defining a plurality of second chambers along the axis, each second chamber intersecting the first chamber and having a fluid therein, the fluid in each second chamber having an agent of a different concentration and/or flowing at a different flow rate; and
a membrane located at an interface region between the first chamber and the plurality of the second chambers, the membrane having cells adhered on a first side facing toward the first chamber and on a second side facing toward the plurality of second chambers, the membrane separating the first chamber from the plurality of the second chambers.

4. The device of claim 3, wherein the cells adhered on the first side include kidney epithelial cells.

5. A device for simulating a function of a tissue, comprising
a first structure defining a first chamber;
a second structure defining a second chamber, the second chamber being coupled to a gradient generator; and
a membrane located at an interface region between the first chamber and the second chamber, the membrane having cells adhered on a first side facing toward the first chamber and on a second side facing toward the second chamber, the membrane separating the first chamber from the second chamber.

6. The device of claim 5, wherein the gradient is continuous or discrete.

7. A method for simulating a function of a tissue, the method comprising:
(a) providing a device, the device comprising:
(i) a first structure defining a first chamber,
(ii) a second structure defining a plurality of second chambers extending along the first chamber, wherein each of the second chambers has a fluid therein, each fluid having an agent of a different concentration, and
(iii) a membrane located at an interface region between the first chamber and the plurality of the second chambers, the membrane having kidney epithelial cells adhered on a first side facing toward the first chamber and on a second side facing toward the plurality of second chambers, the membrane separating the first chamber from the plurality of the second chambers; and
(b) flowing the fluid in the first chamber and the second chambers.

8. The method of claim 7, wherein the fluid in the first chamber and the second chambers are of different flow rates.

9. The method of claim 7, wherein the fluid in each of the second chambers is of a different flow rate.

10. A method for simulating a function of a tissue, the method comprising:
(a) providing a device, the device comprising:
(i) a first structure defining a first chamber along an axis,
(ii) a second structure defining a plurality of second chambers along the axis, each second chamber intersecting the first chamber and having a fluid therein, the fluid in each second chamber having an agent of a different concentration, and
(iii) a membrane located at an interface region between the first chamber and the plurality of the second chambers, the membrane having kidney epithelial cells adhered on a first side facing toward the first chamber and on a second side facing toward the plurality of second chambers, the membrane separating the first chamber from the plurality of the second chambers; and
(b) flowing the fluid in the first chamber and the second chambers.

11. The method of claim 10, wherein the fluid in the first chamber and the second chambers are of different flow rates.

12. The method of claim 10, wherein the fluid in each the second chambers is of a different flow rate.

13. A device for testing agents at different concentrations, the device comprising:
a first structure defining a first chamber;
a plurality of second chambers extending outward along the first chamber, each of the second chambers including a fluid therein and being in fluidic communication with the first chamber, each fluid including an agent of a different concentration; and
a membrane located at an interface region between the first chamber and the plurality of the second chambers, the membrane including cells adhered on a first side facing toward the first chamber and a second side facing toward the plurality of second chambers, the membrane separating the first chamber from the plurality of the second chambers.

14. The device of claim 13, wherein the agents are drugs.

15. The device of claim 13, wherein the cells adhered on the first side are selected from a group consisting of kidney epithelial cells, hepatocytes, and intestinal cells.

16. The device of claim 13, wherein the device is a microfluidic device, the first chamber including a first microfluidic channel, the second chambers being in fluidic communication with the first chamber via second microfluidic channels.

17. A device for testing agents at different concentrations, the device comprising:
a first structure defining a first chamber along an axis;
a plurality of second chambers along the axis, each second chamber intersecting the first chamber and including a fluid therein, the fluid in each second chamber including an agent of a different concentration;
a membrane located at an interface region between the first chamber and the plurality of the second chambers, the membrane including cells adhered on a first side facing toward the first chamber and a second side facing toward the plurality of second chambers, the membrane separating the first chamber from the plurality of the second chambers.

18. The device of claim 17, wherein the cells adhered on the first side are selected from a group consisting of kidney epithelial cells, hepatocytes, and intestinal cells.

19. The device of claim 17, wherein the device is a microfluidic device, the first chamber including a first microfluidic channel, the second chambers being in fluidic communication with the first chamber via second microfluidic channels.

20. A device for exposing cells to gradients, the device comprising:
a first structure defining a first chamber;
a second structure defining a second chamber, the second chamber being coupled to a gradient generator;
a membrane located at an interface region between the first chamber and the second chamber, the membrane including cells adhered on a first side facing toward the first chamber and a second side facing toward the second chamber, the membrane separating the first chamber from the second chamber.

21. The device of claim 20, wherein the cells adhered on the first side are selected from a group consisting of kidney epithelial cells, hepatocytes, and intestinal cells.

22. The device of claim 20, wherein the device is a microfluidic device, the first chamber including a first microfluidic channel, the second chambers being in fluidic communication with the first chamber via second microfluidic channels.

23. A method for testing agents at different concentrations, the method comprising:
(a) providing a device including
(i) a first structure defining a first chamber,
(ii) a plurality of second chambers extending outward along the first chamber, each second chamber of the plurality of second chambers including a fluid therein and being in fluidic communication with the first chamber, each fluid including an agent of a different concentration, and
(iii) a membrane located at an interface region between the first chamber and the plurality of the second chambers, the membrane including cells adhered on a first side facing toward the first chamber and a second side facing toward the plurality of second chambers, the membrane separating the first chamber from the plurality of the second chambers; and
(b) flowing the fluid in the first chamber and the second chambers.

24. The method of claim 23, wherein the fluid in the first chamber and the plurality of second chambers is of different flow rates.

25. The method of claim 23, wherein the fluid in each second chamber is of a different flow rate.

26. The method of claim 23, wherein the device is a microfluidic device, the first chamber including a first microfluidic channel, the plurality of second chambers being in fluidic communication with the first chamber via second microfluidic channels.

27. The method of claim 23, wherein the cells adhered on the first side are selected from a group consisting of kidney epithelial cells, hepatocytes, and intestinal cells.

28. A method for testing agents at different concentrations, the method comprising:
(a) providing a device including
(i) a first structure defining a first chamber along an axis,
(ii) a plurality of second chambers along the axis, each second chamber of the plurality of second chambers intersecting the first chamber and including a fluid therein, the fluid in each second chamber including an agent of a different concentration, and
(iii) a membrane located at an interface region between the first chamber and the plurality of second chambers, the membrane including cells adhered on a first side facing toward the first chamber and a second side facing toward the plurality of second chambers, the membrane separating the first chamber from the plurality of the second chambers; and
(b) flowing the fluid in the first chamber and the second chambers.

29. The method of claim 28, wherein the fluid in the first chamber and the plurality of second chambers is of different flow rates.

30. The method of claim 29, wherein the fluid in each second chamber is of a different flow rate.

31. The method of claim 29, wherein the cells adhered on the first side are selected from a group consisting of kidney epithelial cells, hepatocytes and intestinal cells.

32. The method of claim 28, wherein the device is a microfluidic device, the first chamber including a first microfluidic channel, the second chambers being in fluidic communication with the first chamber via second microfluidic channels.
